(19) United States
(12) Patent Application Publication
(10) Pub. No.: US 2024/0358545 A1
(43) Pub. Date: Oct. 31, 2024

(54) Sterile Dropper Tube

(71) Applicant: Hoffmann Neopac AG, Thun (CH)

(72) Inventors: Philippe Kern, Münsingen (CH); Peter Bossert, Thun (CH); Mario Schuepbach, Konolfingen (CH); Micha Bieri, Steffisburg (CH); Simon Böhlen, Konolfingen (CH)

(73) Assignee: Hoffman Neopac AG, Thun (CH)

(21) Appl. No.: 18/571,849

(22) PCT Filed: Jun. 29, 2022
(86) PCT No.: PCT/EP2022/067841

§ 371 Date: Dec. 19, 2023

(30) Foreign Application Priority Data
Jun. 30, 2021 (WO) PCT/EP2021/068068

(51) Int. Cl. A61F 9/00

(52) U.S. Cl. CPC A61F 9/0026

(57) ABSTRACT

Described is a compressible tube comprising a tube body made of a specific laminate and shoulder and applicator, said applicator being suitable for applying a tube content in dosed droplets and comprises a sterile venting valve. The tube body has a restoring force (R) sufficient to essentially restore the original volume of the tube body after each of a predetermined number of applications. Such tube is especially suitable for the application of ophthalmic sterile drops.

### TECHNICAL FIELD

The present invention relates to sterile dropper containers, especially suitable for ophthalmic preparations.

### BACKGROUND ART

Some pharmaceutical applications, in particular application of ophthalmic preparations, require a high level of accurate dosing of minimal amounts, i.e. few drops, of a pharmaceutical.

Today, eye drops are sold in dropper bottles, e.g. semi-rigid containers. For accurate dropping the preparations must have low viscosity, since otherwise dropping would be affected by the reduced flow of viscous content towards the applicator.

Also already known are eye ointments sold in tubes. However, such ointments cannot be accurately dosed.

Therefore there is a need for a container that allows the accurate dosing also of higher viscosity preparations, especially ophthalmic preparations and that preferably also has good recyclability.

### Abbreviations:

PE=polyethylene
LLDPE=linear low density polyethylene 0.915-0.925 g/cm³
mLLDPE=LLDPE from metallocene based catalyst polymerization route resulting in high density of up to 0.940 g/cm³ while retaining excellent optical properties such as transparency.
LDPE=low density polyethylene 0.910-0.940 g/cm³
HDPE=high density polyethylene 20.941 g/cm³
MDPE=medium density polyethylene 0.926-0.940 g/cm³
PP=polypropylene
EVOH=ethylene vinyl alcohol copolymer
WVTR=water vapor transmission rate
OPP=(mono- or biaxial) oriented polypropylene
BOPP=biaxial oriented polypropylene
AlOx=aluminum oxide
SiOx=silicon oxide

### DISCLOSURE OF THE INVENTION

Where the present description refers to “preferred” embodiments/features, combinations of these “preferred” embodiments/features are also deemed to be disclosed as long as the specific combination of the “preferred” embodiments/features is technically meaningful.

Unless otherwise stated, the following definitions shall apply in this specification:

As used herein, the term “a”, “an”, “the” and similar terms used in the context of the present invention (especially in the context of the claims) are to be construed to cover both the singular and plural unless otherwise indicated herein or clearly contradicted by the context.

As used herein, the term “and/or” means that either all or only one of the elements of said group may be present. For example, “A and/or B” means “only A, or only B, or both A and B”. In the case of “only A”, the term also covers the possibility that B is absent, i.e. “only A, but not B”.

As used herein, the terms “including”, “containing” and “comprising” are used herein in their openended, non-limiting sense. It is understood that the various embodiments, preferences and ranges may be combined at will. Thus, for instance a solution comprising a compound A may include other compounds besides A. However, the term “comprising” also covers, as a particular embodiment thereof, the more restrictive meanings of “consisting essentially of” and “consisting of, so that for instance “a solution comprising A, B and optionally C” may also (essentially) consist of A and B, or (essentially) consist of A, B and C. As used herein, the transitional phrase “consisting essentially of” (and grammatical variants) is to be interpreted as encompassing the recited materials or steps “and those that do not materially affect the basic and novel characteristic(s) of the claimed invention. Thus, the term “consisting essentially of” should not be interpreted as equivalent of “comprising”.

As used herein, the term “about” means that the amount or value in question may be the specific value designated or some other value in its neighborhood. Generally, the term “about” denoting a certain value is intended to denote a range within ±5% of the value. As one example, the phrase “about 100” denotes a range of 100±5, i.e. the range from 95 to 105. Preferably, the range denoted by the term “about” denotes a range within ±3% of the value, more preferably ±1%. Generally, when the term “about” is used, it can be expected that similar results or effects according to the invention can be obtained with in a range of ±5% of the indicated value.

The terms “layer”, “foil” and “film” are used herein interchangeably.

Hence, it is a general object of the invention to provide a dropper container, especially for ophthalmic preparations, that is also suitable for dosing viscous preparations and that preferably also has good recyclability.

Now, in order to implement these and still further objects of the invention, which will become more readily apparent as the description proceeds, the dropper container is manifested by the features that it is a compressible tube comprising a tube body, a shoulder and an applicator, said applicator being suitable for applying a tube content in dosed droplets, said applicator comprising a sterile venting valve and said tube body having a restoring force sufficient to essentially restore the original volume of the tube body after each of a predetermined number of applications.

Other subject matter of the present invention are a method for manufacturing such a compressible tube and its use in combination with a pharmaceutical preparation/composition for ophthalmic use.

“Essentially” with regard to the restoring force means restoring the original volume to at least 80%, preferably at least 85%, more preferred at least 90%, and in particular at least 95%. Such restoring force can be determined by comparing the volume after several applications with the original volume, e.g. by filling the tube body up to the shoulder with a liquid such as water, removing the liquid into a means suitable for volume measuring or weighing and comparing the results before and after use.

In a preferred embodiment, the restoring force is achieved with a body wall that does not comprise an aluminum foil. In particular, the tube body is produced from at least 85%, more preferred at least 90%, most preferred at least 95% polyolefin comprising layers, with other materials optionally present being barrier layers selected from EVOH in a total thickness of 5 to 30 μm, preferably 5 to 15 μm, and/or metal oxide or ceramic layers, in particular AlOx or SiOx nanometer layers in a thickness of <1 μm.

The venting applicator comprises a sterile venting opening, a venting valve that is provided with a sterile filtration means. The respectively equipped venting valve ensures that no contaminants can enter into the tube volume, so that preserving agents can be absent or their amount can at least be significantly reduced and/or the time during which the preparation can safely be used is extended. However, due to the sterile filtration means the tube body needs a higher restoring force than needed for a non-sterile, filter-free venting means.

For good recyclability it is preferred that the whole tube is made of polyolefins, in particular polyethylene and/or polypropylene. In view of the restoring force HDPE and/or PP are preferred, in view of an easy manufacturing process a HDPE rich tube body is preferred. While the tube body can be manufactured by an extrusion or co-extrusion process, presently manufacturing from a laminate is preferred since the flat laminate can easily be printed in line within the low particles, clean room environment demanded for ophthalmic containers. Hence, the tube body described herein does not have an outer surface coated with a sheathing, such as a sheathing made of a polyolefin material, for receiving an imprint. The printed laminates are then formed into a cylinder shaped body and longitudinally seamed. Suitable laminates can be produced by extrusion, co-extrusion of two or more layers, extrusion lamination and/or by lamination using an adhesive.

In this production process, tube shoulders that may have been manufactured (molded) and packaged at another production site, but also in low particles environment, are transported into the low particles environment for the tube production. The laminate, optionally also produced and packaged at a different production site and also in low particles environment, is also transported into the low particles environment for the tube production.

The tube manufacturing process may start with providing the side of the laminate becoming the outside of the tube with an imprint, then forming the laminate into a sleeve and welding the overlapping or abutting ends (blunt welding) of the laminate, thereby generating a longitudinal seam or seal, respectively.

Next, the sleeve is cut to tube bodies and one end of the tube body is provided with a shoulder by welding.

The shoulder is formed such that it sealingly engages with the applicator and provides fixing means for the applicator, i.e. the shoulder may be provided with push-on or screw-on means, dependent on the applicator used.

In a next step, the applicator comprising a closure is connected with the shoulder and the final tube packaged for being sent to the filling station.

As already indicated above, one of the benefits of the tubes of the present invention is their usability in combination with viscous contents due to their form and compressibility/squeezability.

The use of laminates rich in HDPE besides of the good restoring force has the further advantage that HDPE provides low water vapor transmission rate (WVTR) or good barrier properties, respectively. In addition, flexible or compressible or squeezable, respectively, tubes need less material than rigid bottles due to the lower wall thickness of the body.

While monolayer or two layer laminates (the tie layers are not counted herein) can also be used, presently preferred laminates are three layer laminates i.e. laminates composed of three layers or foils or films (the tie layers are not counted herein), composed of an outside layer of foil or film, a center layer or foil or film and an inside (or inner) layer or foil or film that—for improving certain features—may slightly differ in their composition and/or thickness. The layers or foils or films are termed inside and outside with regard to the tube body.

In general these three layers are connected with tie layers that may be thin adhesive layers or extruded layers. Since low molecular weight components are undesired in combination with ophthalmic compositions, it is preferred to laminate the layers by extrusion lamination (also called extrusion coating) and not by an adhesive. In an alternative less preferred embodiment all layers can be coextruded.

The inside layer and the outside layer preferably comprise a low amount of LDPE or LLDPE including mLLDPE for improved sealability. In case of blunt welding, the seam is strengthened by means of a sealing band along the seam. Generally, this sealing band is of LDPE or LLDPE comprising PE material.

The composition of the outside layer is e.g. improved for sealability to the inside layer and/or the shoulder and also for printability. It may also be provided with a light barrier additive or light blocking additive for protecting the content of the tube, such as TiO₂, and/or other pigments for esthetic purposes and/or light (UV/VIS) stabilizers, such as UV absorbers, for protecting the laminate during the in-use shelf-life of the compressible tube.

The inner or inside layer, in contact with the content, may be of higher quality than the other layers, e.g. at least in part of pharmaceutical grade material, and also improved for sealability to the outside layer and/or the shoulder and/or with itself.

Each of the layers, in particular the center layer and/or the inside or inner layer can further be provided with a barrier layer. The inside or inner layer may preferably be provided with a sandwiched EVOH layer, while a PE center layer/foil may e.g. comprise a metallization and an oriented PP center film, such as an OPP or a BOPP center (carrier) film may be provided with an SiOx or AlOx layer and/or a metallization. Monoaxial or biaxial oriented foils can also be used as center layer without barrier layer, although their advantage is limited since no oxygen barrier is needed. In some cases they may add to the desired mechanical properties.

### BRIEF DESCRIPTION OF THE DRAWINGS

The invention will be better understood and objects other than those set forth above will become apparent when consideration is given to the following detailed description thereof. Such description makes reference to the annexed drawings, wherein:

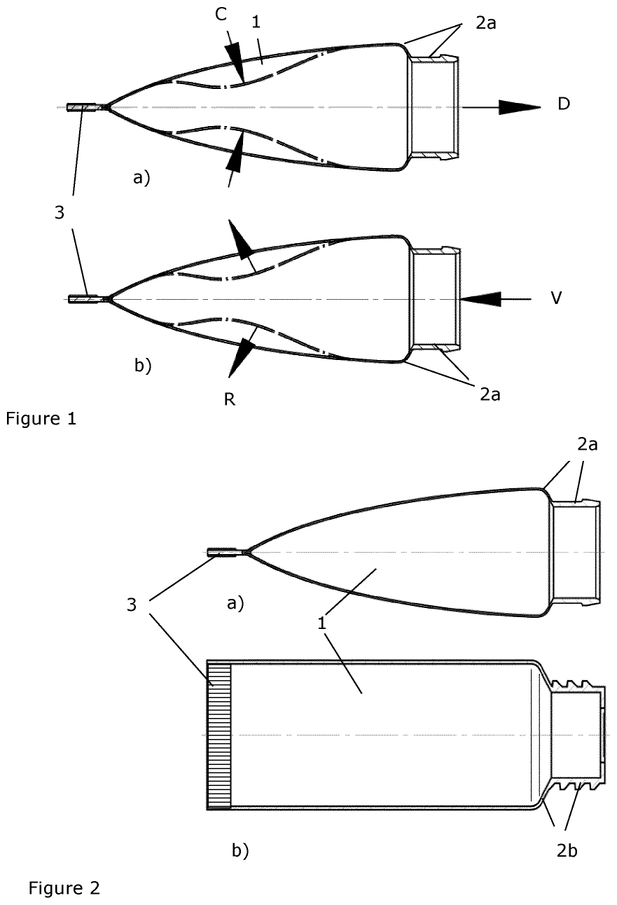

FIG. 1 visualizes the restoring force, i.e.
a) shows the direction of the compressing/squeezing force C applied upon withdrawing content by dropping D,
b) shows the direction of the restoring force R upon ventilation V.

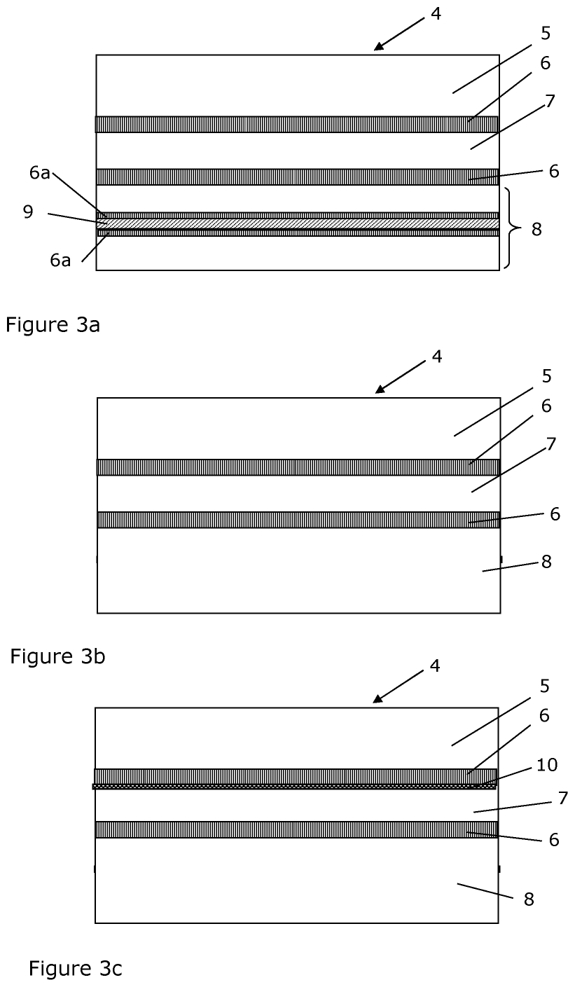

FIG. 2 shows two types of shoulders with different means for attaching the applicator, with
a) showing a push-on shoulder and
b) showing a screw-on shoulder.

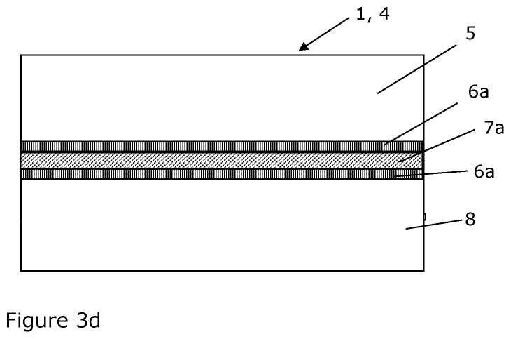

FIG. 3 schematically shows three kinds of laminates and one co-extruded tube body wall, wherein
a) shows a laminate or a laminate based tube body with HDPE center layer with HDPE/LDPE or HDPE/LLDPE outside layer, and a HDPE/LDPE or HDPE/LLDPE inside layer with coextruded EVOH intermediate layer, all layers connected with tie layers for improved stability,
b) shows a laminate as in a) but without co-extruded EVOH intermediate layer,
c) shows a laminate or a laminate based tube body with a center layer provided with a functional barrier layer such as an SiOx layer or an AlOx layer (although not shown, the inside layer may also be provided with an intermediate EVOH layer),
d) shows an extruded tube body with a co-extruded EVOH barrier layer as center layer and two coextruded tie layers for obtaining improved adhesion between EVOH and PE.

### MODES FOR CARRYING OUT THE INVENTION

Applicators or droppers with a sterile ventilation valve and suitable for dosing single drops of constant size upon constant pressure are known and are e.g. obtainable from the firms Silgan, Nemera or Aptar.

In the manufacturing of sterile dropper tubes with a tube body formed from a laminate, the laminate and the tube shoulder can be manufactured in another facility, provided that they are produced and packaged in low particle (clean room) environment.

The actual tube forming method in a low particle environment (production in clean room classification ISO 7 or better) starts with in line printing of the laminate. In line printing of the laminate is advantageous since no rolling of the laminate is needed and therefore no ink transfer to the backside (inside of tube) can occur.

Printing is followed by sleeve forming and welding overlapping regions or abutting edges (blunt welding), optionally provided with a sealing band. Presently preferred are overlapping seams.

Then the sleeve is cut into tube bodies 1 of desired length and provided with a tube shoulder 2a, 2b at one end.

FIGS. 1 and 2 show the tube body 1 with shoulder 2a, 2b but without applicator. The applicator is such that it has a groove into which the shoulder 2a, 2b engages, i.e. the applicator extends on the interior and the exterior surfaces of the shoulder 2a, 2b.

Once the tube body is filled with the content, the end of the tube body opposite the shoulder/applicator is sealed. This seal 3 is also termed end seal or end seam 3. For stability reasons, it proved advantageous to position the longitudinal seam extending decentralized from the end seam 3 to the shoulder 2a, 2b and not from its center or middle, respectively.

The tube body described herein does not have an outer surface coated with a sheathing, such as a sheathing made of a polyolefin material, for receiving an imprint.

Upon use, compression/squeezing pressure C is applied to the tube body and content drops out D (see FIG. 1a). As soon as the compressing/squeezing pressure is released, the restoring force R sucks in air through the applicator V until the original tube volume is regenerated (see FIG. 1b).

As shown in FIG. 2, the shoulder in addition is provided with fixing means for the applicator such as push-on means 2a or screw-on means 2b.

The applicator as bought has a sterile ventilation valve, i.e. an opening provided with a filtering means, and in general is also provided with a cap that can either allow access of air to the venting valve, or seal the venting valve.

Presently preferred are caps that do not seal the venting valve. The longtime access of the venting valve allows full restoration of the original volume even if the restoration takes some time. In addition, it allows the user to mount the cap directly after use, i.e. without waiting for restoration.

For viscous contents, the cap should be such that the tube can be placed on the cap to ensure that the content flows towards the applicator between applications, thereby ensuring sufficient content in place for the dosed application.

Using a compressible tube instead of a bottle for dropping ophthalmic pharmaceuticals has several ad-vantages, e.g.
It can be used to dose a variety of liquids from low viscosity liquids of about 1 mPas up to viscous liquids of up to 2000 mPas.
It has better water vapor transmission rate (WVTR) barrier properties.
Due to the sterile venting valve in the applicator the content needs less or no preservatives and the content can longer be used.
It needs less material compared with a bottle.

For a reliable dosing of a few drops over the envisaged usable time of the content, a constant and sufficiently high restoring force R is important.

The minimal restoring force required is dependent on the sterile ventilation valve of the applicator used and in particular its sterile filtration means. Such sterile filtration means can be a filter material or a suitably shaped access between the outer and inner surface of the applicator.

While no oxygen barrier is needed due to the desired ventilation, the WVTR needs to be minimized since constant weight/viscosity is important for accurate dosing over the whole lifetime.

For recyclability it is preferred that the whole tube is made of polyolefin. While polyolefins with the same monomer units are preferred, i.e. polyethylene (PE) or polypropylene (PP), most of the applicators presently available are PP based while many tubes are preferably made of PE. Presently preferred are laminates with a high content of high density polyethylene (HDPE), option-35 ally and preferably admixed with minor amounts of low density polyethylene (LDPE) or linear low density polyethylene (LLDPE) for optimizing specific features such as sealability.

The tube body 1 may be made by extrusion or co-extrusion (FIG. 3d). However, presently preferred is a tube manufactured from a laminate 4.

For recyclability, it is preferred that the tubes are free of aluminum foils. The tube body may comprise barrier layers (7a, 9, 10) of materials other than polyolefins, said barrier layers (7a, 9, 10) being selected from the group consisting of EVOH layers, metal oxide layers, ceramic layers, thin metal layers (metallizations) and combinations thereof. While thin film metallizations are acceptable as long as included into the laminate structure and not being a surface metallization which could lead to NIR (near infrared) sorting issues in mechanical recycling streams, it is preferred that any barrier layer (7a, 9, 10) is either an EVOH layer (7a, 9), or a thin metal and/or metal oxide or ceramic layer (10), such as an AlOx or SiOx layer. While a metallization may be applied on a PE film, for AlOx and SiOx an oriented PP center (Carrier) layer is preferred.

The compressible tube composed of tube body, shoulder and applicator is preferably made of polyolefin materials to at least 90%, preferably at least 95% more preferably about 98%, in particular polyolefin materials selected from polyethylene and/or polypropylene.

Where the tube body comprises an EVOH barrier layer, such layer preferably is limited to at most 10% of the body wall thickness. Where an EVOH barrier layer is present in the tube body, the EVOH content of the tube body wall is limited to layers of a thickness of at most 30 μm, more preferred at most 20 μm, most preferred about 9 μm. Preferably, the tube body and the laminate claimed and described herein contain one EVOH barrier layer of a thickness of at most 30 μm, more preferred at most 20 μm, most preferred about 9 μm.

In particular, the tube body is produced from at least 85% polyolefin comprising layers, preferably polyolefin comprising layers made up of the same monomer units (PE) or with minor amounts of compatible monomer units, like some ethylene units comprised in PP, or the maleic anhydride grafted LLDPE tie layers 6a. Other materials optionally present are selected from EVOH barrier layers, in general EVOH layers of a thickness of at most 30 μm, more preferred at most 20 μm, most preferred about 9 μm and/or metal oxide or ceramic layers, in particular AlOx or SiOx nanometric layers in a thickness of <1 μm.

If an EVOH layer is present, also tie layers (6a) of maleic anhydride grafted LLDPE are present between EVOH and PE in a thickness each that is similar to or up to about 50% smaller than the thickness of the EVOH layer. Preferably, the tie layer has a thickness of about 6 μm to about 9 μm, more preferably from about 7 μm about 9 μm.

Presently preferred is a laminate 4 of three layers or films or foils, referred to as outside 5, center 7 and inside 8 layers, films or foils (for these lay-ers/films/foils these terms are used interchangeably, i.e. as synonyms). These foils 5, 7, 7a, 8 are coextruded or connected with each other by means of an extruded tie layer 6, 6a. Instead of the extruded tie layer 6 also an adhesive might be used. However, an adhesive is less preferred since low molecular weight ingredients might mi-grate into the content.

The foils may be PP or rich in HDPE. For foils rich in HDPE, the following preferences exist:
The HDPE content in the HDPE based material should at least be 70%, preferably at least 80%.

The outside foil 5 in general contains at least about 85% polyethylene composed of HDPE and LDPE or HDPE and LLDPE, wherein the HDPE content is at least about 70%, preferably at least about 80%, more preferably at least about 85%, much preferably about 90%. In addition, it may comprise up to 15% additives and/or pigments for esthetic purposes. Preferably the additives are selected from light barrier or light blocking additives, such as TiO₂ for improved light shielding of the content, and light (UV/VIS) stabilizers, such as UV absorbers. The light barrier or light blocking additives provide light protection for content of the tube, while the light (UV/VIS) stabilizers ensure that the laminate is light protected during the in-use shelf-life of the compressible tube. Preferably the outside layer has a thickness from about 80 μm to about 140 μm, more preferably from about 100 μm to about 140 μm, much preferably from about 110 μm to about 130 μm, such as about 120 μm. The outside foil described herein is preferably a coextruded foil containing at least about 85% polyethylene composed of HDPE and LDPE or HDPE and LLDPE, wherein the HDPE content is at least about 70%, preferably at least about 80%, more preferably at least about 85%, much preferably about 90%. For example, the outside foil may be a three layers coextruded foil, a four layers coextruded foil, a five layers coextruded foil, a six layers coextruded foils, or a seven layers coextruded foil. The layers contained by the three layers coextruded foil, the four layers coextruded foil, the five layers coextruded foil, the six layers coextruded foils, or the seven layers coextruded foil may have the same composition (e.g. 80/20 HDPE/LLDPE), or may have a different composition (e.g. a three layers coextruded foil, wherein a first layer contains 80% HDPE and 20% LLDPE, a second layer contains 85% HDPE, 10% LLDPE and 5% additives and/or pigments for esthetic purposes, and a third layer contains 90% HDPE, 7% LLDPE and 3% additives). The layers contained by the three layers coextruded foil, the four layers coextruded foil, the five layers coextruded foil, the six layers coextruded foil, and the seven layers coextruded foil may have the same or a different thickness.

The center foil 7 might be a monoaxial or biaxial oriented foil because such foils provide higher mechanical strength. For example, the center foil may be an oriented polypropylene foil, such as an OPP or a BOPP foil, optionally provided with a barrier layer selected from an SiOx or an AlOx layer and/or a metallization. However, a non-oriented HDPE rich laminate is preferred, in particular an at least 90%, preferably 100% HDPE foil, optionally provided with a barrier coating 10 such as a metallization. In an preferred embodiment, the center layer or foil or film 7 is a non-oriented HDPE layer or foil or film containing at least about 85%, preferably at least about 90% HDPE, more preferably 100% HDPE. The center foil 7 is optionally provided with a barrier coating (10) such as a metallization (thin metal layer). Preferably, the center foil is not provided with a barrier coating (10), such as metallization. The center foil may contain additives as described herein and/or pigments for esthetic purposes as described herein. Preferably the additives are selected from light barrier or light blocking additives for providing light protection to the tube content, and light (UV/VIS) stabilizers, such as UV absorbers, for protecting the tube laminate during the in-use shelf-life storage. Preferably the center layer has a thickness from about 60 μm to about 100 μm, more preferably from about 70 μm to about 90 μm, such as about 80 μm. The center foil described herein is preferably a coextruded non-oriented HDPE layer or foil or film containing at least about 85%, preferably at least about 90% HDPE, more preferably 100% HDPE. For example, the center foil may be a three layers coextruded foil, a four layers coextruded foil, a five layers coextruded foil, a six layers coextruded foil, or a seven layers coextruded foil. The layers contained by the three layers coextruded foil, the four layers coextruded foil, the five layers coextruded foil, the six layers coextruded foil, and the seven layers coextruded foil may have the same composition (e.g. 90% HDPE and 10% additives and/or pigments for esthetic purposes), or may have a different composition (e.g. a three layers coextruded foil, wherein a first layer contains 90% HDPE and 10% additives and/or pigments for esthetic purposes, a second layer contains 95% HDPE and 5% additives and/or pigments for esthetic purposes, and a third layer contains 100% HDPE). The layers contained by the three layers coextruded foil, the four layers coextruded foil, the five layers coextruded foil, the six layers coextruded foil, and the seven layers coextruded foil may have the same or a different thickness.

The inside or inner foil 8 can be a mono foil or a coextruded foil, e.g. comprising a coextruded EVOH barrier layer 9. In the case of a mono foil, the preferred material is HDPE/LDPE or HDPE/LLDPE foil with a HDPE content of at least 70% for improved sealability. Preferably, the inside foil 8 is a coextruded foil comprising a coextruded EVOH layer. Such coextruded foil comprising a coextruded EVOH layer contains at least about 70% polyethylene of the same composition as the mono foil (i.e. at least about 70% polyethylene composed of HDPE and LDPE and/or HDPE and LLDPE, wherein the HDPE content is at least about 70%). Hence, in case of a coextruded foil containing an EVOH layer, the composition of the polyethylene is the same as in the mono foil, but an EVOH layer, as well as tie layers (6a) of maleic anhydride grafted LLDPE arranged between the EVOH layer and the PE layer are present. The EVOH layer has a thickness of at most 30 μm, preferably of at most 20 μm, most preferably of about 9 μm. Each of the tie layers 6a has a thickness similar to or up to about 50% smaller than the thickness of the EVOH layer. The inner layer has preferably a thickness from about 80 μm to about 120 μm, more preferably from about 90 μm to about 110 μm, such as 100 μm. Preferably, the coextruded foil (8) comprising a co-extruded EVOH layer has the following structure:
polyethylene layer composed of HDPE and LDPE and/or HDPE and LLDPE, wherein the HDPE content is at least about 70%,
tie layer of maleic anhydride grafted LLDPE (6a),
EVOH layer (9),
tie layer of maleic anhydride grafted LLDPE (6a),
polyethylene layer composed of HDPE and LDPE and/or HDPE and LLDPE, wherein the HDPE content is at least about 70%.

Each of the tie layers 6 between inside, center and outside foil contains at least about 90% HDPE. The tie layer may have a higher HDPE content, such as higher than 95%, preferably 100% HDPE. The tie layers 6 may contain additives as described herein and/or pigments for esthetic purposes as described herein. Preferably the additives are selected from light barrier or light blocking additives for providing light protection to the tube content, and light (UV/VIS) stabilizers, such as UV absorbers, for protecting the tube laminate during the in-use shelf-life storage. Preferably, each of the tie layers is a mono foil or mono layer or mono film. Their thickness ranges from about 10 to about 30 μm, preferably from about 20 to about 30 μm.

A preferred embodiment according to the present invention is directed to a compressible tube as described herein, wherein the tube body is a laminate (4) composed of three layers or foils or films (5, 7, 8), an outside layer or foil or film (5), a center layer or foil or film (7), and an inner layer or foil or film (8), wherein
the outside layer or foil or film (5) contains at least about 85% polyethylene composed of HDPE and LDPE, or HDPE and LLDPE, wherein the HDPE content is at least about 70%, preferably at least about 80%, more preferably at least about 85%, much preferably about 90% and the outside layer or foil or film (5) optionally comprises up to 15% additives and/or pigments for esthetic purposes, wherein the additives are preferably selected from light barrier additives and light stabilizers,
the center layer or foil or film (7) is a non-oriented HDPE layer or foil or film containing at least 85%, preferably at least 90% HDPE, and
the inner layer or film or foil (8) is a mono foil or a coextruded foil comprising a coextruded EVOH barrier layer (9), preferably a coextruded foil comprising a coextruded EVOH barrier layer (9), wherein
the mono layer or film or foil is a HDPE/LDPE or HDPE/LLDPE film or foil with a HDPE content of at least 70%, and
the coextruded layer or film or foil comprising an EVOH layer comprises at least 70% polyethylene of the same composition as the mono foil and an EVOH layer of a thickness of at most 30 μm, more preferred at most μm, most preferred about 9 μm, as well as tie layers (6a) of maleic anhydride grafted LLDPE arranged between 20 the EVOH layer and a polyethylene layer in a thickness each that is similar to or up to about 50% smaller than the thickness of the EVOH layer. In the compressible tube claimed and described herein and the laminate claimed and described herein, preferably the outside layer or foil or film (5) and the center layer or foil or film (7) are connected via a tie layer (6) as described herein and the center layer or foil or film (7) and the inner layer or foil or film (8) are also connected via a tie layer (6) as described herein. The tie layer (6) is preferably an extruded tie layer. Preferably, the tie layer (6) contains at least about 90%, preferably about 958, more preferably 100% HDPE. Preferably, the outside foil and the center foil are coextruded foils as described herein.

An alternative embodiment relates to a compressible tube as described herein, wherein the tube body is a laminate composed of three layers or foils or films, an outside foil or film, a center foil or film and an inner foil or film, wherein
the outside foil or film is a mono foil or a coextruded foil comprising a coextruded EVOH barrier layer,
wherein
the mono film or foil is a HDPE/LDPE or HDPE/LLDPE film or foil with a HDPE content of at least 70%, and
the coextruded film or foil comprising an EVOH layer comprises at least 70% polyethylene of the same composition as the mono foil and an EVOH layer of a thickness of at most 30 μm, more preferred at most 20 μm, most preferred about 9 μm, as well as tie layers of maleic anhydride grafted LLDPE arranged between the EVOH layer and a polyethylene layer in a thickness each that is similar to or up to about 50% smaller than the thickness of the EVOH layer,
the center foil or film is a non-oriented HDPE foil or film containing at least 85%, preferably at least 90% HDPE, and
the inner foil or film contains at least about 85% polyethylene composed of HDPE and LDPE or HDPE and LLDPE, wherein the HDPE content is at least about 70%, preferably at least about 80%, more preferably at least about 85%, much preferably about 90% and the outside foil or film. The outside foil or film and the center foil or film are preferably connected via a tie layer and the center foil or film and the inner foil or film are also connected via a tie layer. The tie layer is preferably an extruded tie layer. Preferably, the tie layer contains at least about 90%, preferably about 95%, more preferably 100% HDPE and has a thickness from about 10 to about 30 μm, preferably from about 20 to about 30 μm. The outside foil has preferably a thickness from about 80 μm to about 120 μm, more preferably from about 90 μm to about 110 μm, such as 100 μm. Preferably the center foil has a thickness from about 60 μm to about 100 μm, more preferably from about 70 μm to about 90 μm, such as about 80 μm. Preferably the inner foil has a thickness from about 80 μm to about 140 μm, more preferably from about 100 μm to about 140 μm, much preferably from about 110 μm to about 130 μm, such as about 120 μm.

In case of a coextruded laminate 4 or tube body 1, the center layer may be an EVOH layer 7a sandwiched between two tie layers 6a and inside 8 and outside 5 layers. Such a coextruded laminate 4 or tube body 1 is shown in FIG. 3d.

It has been found that the restoring force R is dependent on the laminate thickness, the HDPE content and the laminate structure. Thus, if the restoring force is insufficient, the layer thickness and/or the HDPE content may be enhanced and/or the HDPE type and/or the laminate structure may be adapted.

HDPE is a presently preferred material because it has good water vapor barrier properties, can be processed on usual PE tube manufacturing lines and provides good restoring force.

Suitable tube dimensions range from diameters from 16 mm to 30 mm and volumes from 5 ml to 100 ml.

For ophthalmic preparations and available applicators presently a volume of 10 ml and a diameter of 22 mm is preferred.

For such a tube a minimal restoring force of 63 mbar proved sufficient. Lower restoring forces led to a reduced number of accurate doses. Long lasting restoring force was e.g. obtained with a laminate with the following layers/thicknesses:
outside layer 5/120 μm, tie layer 6/25 μm, center layer 7/80 μm, tie layer 6/25 μm, inside layer 8/100 μm.

With a thinner laminate composed of outside layer 5/80 μm, tie layer 6/20 μm, center layer 7/80 μm, tie layer 6/20 μm, inside layer 8/100 μm few samples did not fully achieve the 63 mbar.

As already indicated above, for good sealability the inside and the outside foils comprise LDPE and/or LLDPE. Presently preferred materials are:
Outside layer/film/foil 5: 90/10 HDPE/LDPE or HDPE/LLDPE
Center layer/film/foil 7: 100 HDPE
Inside layer/film/foil 8: 80/20 HDPE/LDPE or
HDPE/LLDPE, or a foil with HDPE/LLDPE 80/20 inside/outside plus 9 μm EVOH and 2×7 μm tie layer 6a (maleic anhydride grafted LLDPE)
Tie layer 6: 100 HDPE

The present invention may be further summarized by reference to the following clauses #1-#15:

#1. Compressible tube comprising a tube body (1), a shoulder (2a, 2b) and an applicator, said applicator being suitable for applying a tube content in dosed droplets, said applicator comprising a sterile venting valve and said tube body having a restoring force (R) sufficient to essentially restore the original volume of the tube body after each of a predetermined number of applications.

#2. The compressible tube of #1, wherein the restoring force (R) is such that the original volume is restored to at least 85%, preferably at least 90%, more preferred at least 95% for the number of applications possible until the container is empty.

#3. The compressible tube of #1 or #2, wherein the whole tube is made of polyolefin materials to at least 90%, preferably at least 95% more preferred about 98%, in particular polyolefin materials selected from polyethylene and/or polypropylene.

#4. The compressible tube of any one of #1-#3, wherein tube body comprises barrier layers (7a, 9, 10) of materials other than polyolefins, said barrier layers (7a, 9, 10) being selected from the group consisting of EVOH layers, metal oxide layers, ceramic layers, metallizations and combinations thereof.

#5. The compressible tube of claim #4, wherein the EVOH content of the tube body wall is limited to layers of a thickness of at most 30 μm, more preferred at most 20 μm, most preferred about 9 μm.

#6. The compressible tube of any one of #1-#5, wherein the tube body is a laminate composed of three layers or foils or films (5, 7, 8), an outside layer or foil or film (5), a center layer or foil or film (7) and an inner layer or foil or film (8), wherein preferably the outside layer or foil or film (5) and the center layer or foil or film (7) are connected via a tie layer (6, 6a) and the center layer or foil or film (7) and the inner layer or foil or film (8) are also connected via a tie layer (6, 6a).

#7. The compressible tube of claim #6, wherein the HDPE content of the tube body is at least 70% and/or
the outside layer or foil or film (5) is at least 85% polyethylene composed of HDPE and LDPE or HDPE and LLDPE, wherein the HDPE content is at least 70%, preferably about 90% and the outside layer or foil or film (5) optionally comprises up to 15% additives such as TiO₂ for improved light shielding of the content, or it may comprise pigments for esthetic purposes, and/or
the polyolefin of the center layer or film or foil (7) is at least 90% HDPE, optionally provided with a barrier coating (10) such as a metallization, or
the polyolefin of the center layer is a mono-directional or bi-directional oriented polyethylene or polypropylene foil or film (7) provided with a barrier coating (10) that is AlOx or SiOx and optionally a metallization, and/or
the inside layer or film or foil (8) is a mono foil or a coextruded foil, e.g. comprising a coextruded EVOH barrier layer (9), wherein the mono layer or film or foil is a HDPE/LDPE or HDPE/LLDPE foil with a HDPE content of at least 70%, and wherein a coextruded layer or film or foil comprising an EVOH layer comprises at least 70% polyethylene of the same composition as the mono foil and EVOH layers of a thickness of at most 30 μm, more preferred at most 20 μm, most preferred about 9 μm, as well as tie layers (6a) of maleic anhydride grafted LLDPE between EVOH and PE in a thickness each that is similar to or up to about 50% smaller than the thickness of the EVOH layer, and/or
at least one tie layer (6) of >95, preferably 100% HDPE.
#8. The compressible tube of any one of #1 to #7, wherein the tube body is a coextruded laminate and the center layer (7, 7a) is an EVOH layer (7a) sandwiched between inside layer (8) and outside layer (5) and connected thereto via two tie layers (6a).

#9. The compressible tube of any of #1 to #7, wherein the laminate (4) has a structure of
outside layer/film (5) in a thickness of 100-140 μm, in particular about 120 μm
tie layers (6) in a thickness of 20-30 μm, in particular about 25 μm
center layer/film (7) in a thickness of 60-100 μm, in particular about 80 μm
tie layer (6) in a thickness of 20-30 μm, in particular about 25 μm
inside layer/film (8) in a thickness of 80-120 μm, in particular about 100 μm
   and a layer composition of
outside layer/film (5) 90/10 HDPE/LDPE or HDPE/LLDPE
center layer/film (7) 100 HDPE
   inside layer/film (8) 80/20 HDPE/LDPE or HDPE/LLDPE, or a layer/film with HDPE/LLDPE 80/20 in-side/outside plus 9 μm EVOH layer (9) and 2×7 μm tie layer (6a)
tie layer (6) 100 HDPE.

#10. The compressible tube of any of #1-#9, wherein the longitudinal seam of the tube body extends decentralized from an end seam (3) to the shoulder (2a, 2b).

#11. The compressible tube of any of #1-#10, wherein the container has a volume of 10 ml, a diameter of 22 mm and the restoring force is 63 mBar.

#12. The compressible tube of any of #1-#11, wherein the applicator has a vented cap.

#13. The compressible tube of any of #1-#12 that contains a pharmaceutical composition for ophthalmic use.

#14. A laminate (4) having the following structure
outside layer/film (5) in a thickness of 100-140 μm, in particular about 120 μm
tie layers (6) in a thickness of 20-30 μm, in particular about 25 μm
center layer/film (7) in a thickness of 60-100 μm, in particular about 80 μm
tie layer (6) in a thickness of 20-30 μm, in particular about 25 μm
inside layer/film (8) in a thickness of 80-120 μm, in particular about 100 μm
   and a layer composition of
outside layer/film (5) 90/10 HDPE/LDPE or HDPE/LLDPE
center layer/film (7) 100 HDPE
inside layer/film (8) 80/20 HDPE/LDPE or HDPE/LLDPE, or a layer/film with HDPE/LLDPE 80/20 inside/outside plus 9 μm EVOH layer (9) and 2×7 μm tie layer (6a)
tie layer (6) 100 HDPE

#15. Use of a compressible tube of any of #1-#12 for being filled with a pharmaceutical composition for ophthalmic use.

### Experimental Part:

Tubes with diameter of 22 mm and volume of 10 ml were produced as indicated above. The tube body was either made of a laminate composed of
A) outside layer/80 μm, tie layer/20 μm, center layer/80 μm, tie layer/20 μm, inside layer/100 μm, or
B) outside layer/120 μm, tie layer/25 μm, center layer/80 μm, tie layer/25 μm, inside layer/100 μm.

Both laminates were made with foils/layers of the following composition:
Outside foil 5: 90/10 HDPE/LDPE
Center foil 7: 100 HDPE
Inside foil 8: barrier foil composed of
HDPE/LLDPE 80/20 inside/outside plus 9 μm EVOH layer 9 and 2×7 μm tie layer 6a (maleic anhydride grafted LLDPE)
Tie layer 6: 100 HDPE

The applicator used was a push-on applicator obtainable from Aptar.

While usually oxygen transmission is relevant for tubes, this measure is irrelevant for the present, vented tubes.

The above described tubes were tested for their initial restoring force, for the conservation of the restoring force over multiple applications, the WVTR barrier effect and the weight loss upon storing.

The restoring force was tested as indicated in FIG. 1 with a) showing the compression, the squeezing situation, b) the restoring situation.

In this test, the open end/applicator end of a squeezed tube is sealingly placed on a vacuum measuring device to detect the vacuum (sucking force) of the tube. The results obtained are shown in Table 1.

TABLE 1

| | | A) | | B) |
|---|---|---|---|---|
| | | Thickness of body wall | | |
| | | m0.3 mm | s0.3 mm | s0.35 mm |
| minimal force | | 63 | 63 | 63 |
| Variability | + | 60 | 60 | 60 |
| | − | 0 | 0 | 0 |
| Sample | 1 | 75 | 74 | 82 |
| | 2 | 50 | 67 | 86 |
| | 3 | 55 | 64 | 80 |
| | 4 | 68 | 73 | 88 |
| | 5 | 58 | 75 | 84 |
| | 6 | 61 | 76 | 90 |
| | 7 | 54 | 73 | 85 |
| | 8 | 60 | 69 | 83 |
| | 9 | 73 | 60 | 79 |
| | 10 | 64 | 68 | 82 |

| | | | |
|---|---|---|---|
| MIN | 50 | 60 | 79 |
| MAX | 75 | 76 | 90 |
| ø | 62 | 70 | 84 |
| Median | 61 | 71 | 84 |

The letters m and s before the wall thickness indicate whether the longitudinal seam extended from the middle of the end seam (m) or decentralized to a side(s).

The minor results obtained with tubes with a seam extending from the middle of the end seam were due to bending of the tube. Such bending could be eliminated by positioning the longitudinal seam at one of the sides.

Table 2 shows the squeezing and restoring forces over a multitude of applications. From each tube 3 times a day 4 drops were dosed and the squeezing force/restoring force indicated.

The following kinds of tubes were tested and the mean value per dosing indicated.

BB (laminate B, vented cap, content physiological NaCl solution)

BK (laminate B, sealing cap, content physiological NaCl solution)

FB (laminate B, vented cap, content adjusted to 1000 mPas)

GB (laminate B, vented cap, content adjusted to 1000 mPas) (produced in a second facility)

HB (laminate B, vented cap, physiological NaCl solution) (produced in a second facility)

JB (laminate B, vented cap, content adjusted to 1000 mPas) (produced in a third facility)

KB (laminate B, vented cap, content adjusted to 1000 mPas) (produced in a third facility

In the following table the number of samples is indicated after the sample designation.
0=no deformation
1=slight remaining deformation
2=stronger deformation, force had to be slightly enhanced
e=tube fully emptied

TABLE 2

| Day | BB/10 | BK/10 | FB/4 | GB/10 | HB/5 | JB/10 | KB/10 |
|---|---|---|---|---|---|---|---|
| 1 | 30 × 0 | 30 × 0 | 0 | 0 | 0 | 0 | 0 |
| 2 | 30 × 0 | 30 × 0 | 0 | 0 | 0 | 0 | 0 |
| 3 | 30 × 0 | 3 × 1 | 0 | 0 | 0 | 0 | 0 |
| 4 | 30 × 0 | 16 × 1 | 0 | 0 | 0 | 0 | 0 |
| 5 | 30 × 0 | 18 × 1 | 0 | 2 × 1 | 0 | 3 × 1 | 6 × 1 |
| 6 | 15 × 1 | 24 × 1 | 0 | 2 × 1 | 0 | 3 × 1 | 6 × 1 |
| | | | | 2 × 2 | | | |
| 7 | 19 × 1 | 24 × 1 | 0 | 7 × 1 | 3 × 1 | 6 × 1 | 6 × 1 |
| | | | | 2 × 2 | | | |
| 8 | 21 × 1 | 24 × 1 | 1 × 1 | 6 × 1 | 3 × 1 | 5 × 1 | 4 × 1 |
| | | | 1e | 3 × 2 | | 1 × 2 | 2 × 2 |
| | | | | 3e | | | |
| 9 | 21 × 1 | 24 × 1 | 3 × 1 | 0 or 1 | 6 × 1 | 3 × 1 | 3 × 1 |
| | | | | 5e | | 3 × 2 | 3 × 2 |
| 10 | 24 × 1 | 26 × 1 | 1 × 1/3e | 2e | 6 × 1 | 3 × 1 | 3 × 1 |
| | | | | | | 3 × 2 | 3 × 2 |
| 11 | 24 × 1 | 27 × 1 | | | 7 × 1 | 4 × 1 | 4 × 1 |
| | | | | | | 2 × 2 | 2 × 2 |
| 12 | 27 × 1 | 30 × 1 | | | 9 × 1 | 6 × 1 | 6 × 1 |
| 13 | 30 × 1 | 28 × 1 | | | 9 × 1 | 6 × 1 | 6 × 1 |
| | | 2 × 2 | | | | | |
| 14 | 30 × 1 | 30 × 1 | | | 13 × 1 | 6 × 1 | 5 × 1 |
| | | | | | | | 1 × 2 |
| 15 | 30 × 1 | 30 × 1 | | | 15 × 1 | 5 × 1 | 3 × 1 |
| | | | | | | 7e | 1 × 2 |
| | | | | | | | 8e |
| 16 | 24 × 1 | 30 × 1 | | | 15 × 1 | 3e | 2e |
| 17 | 24 × 1 | 30 × 1 | | | 12 × 1 | | |
| | | | | | 3 × 2 | | |
| 18 | 25 × 1 | 30 × 1 | | | 12 × 1 | | |
| | | | | | 3 × 2 | | |
| 19 | 24 × 1 | 30 × 1 | | | 12 × 1 | | |
| | | | | | 3 × 2 | | |
| 20 | 24 × 1 | 30 × 1 | | | 12 × 1 | | |
| | | | | | 3 × 2 | | |
| 21 | 24 × 1 | 30 × 1 | | | 12 × 1 | | |
| | | | | | 3 × 2 | | |
| 22 | 24 × 1 | 30 × 1 | | | 6 × 1 | | |
| | | | | | 3 × 2 | | |
| | | | | | 3e | | |
| 23 | 7e | 1e | | | 2e | | |
| 24 | 1e | 8e | | | | | |
| 25 | 2e | 1e | | | | | |

As can be seen from the above table, all vented tube samples with laminate B) provided good results even with viscous content until the tube was fully emptied.

A further important feature for reliable dosing is the WVTR barrier effect or the weight conservation, respectively.

WVTR barrier measurements after 24 h at 40° C. and 75% rh are shown in Table 3 below:

TABLE 3

| | WVTR mean | standard |
|---|---|---|
| Sample | [g/(pkg*Tg)] | deviation |
| Tube made with laminate A) | 0.00109 | 0.00002 |
| Tube made with laminate B) | 0.00103 | 0.00009 |
| Reference, bottle with same | 0.00146 | 0.00003 |
| applicator 21 × 38 × 1 mm | | |

The applicator used in the above measurements was supplied by Aptar.

Also weight loss experiments have already been performed at 40° C. (Table 4) and at room temperature (RT) (Table 5)

TABLE 4
(40° C. at 20% rh):

| | | 7 | 14 | 1 | 3 | extrapolation |
|---|---|---|---|---|---|---|
| Sample | | days | days | month | months | 6 months: |
| laminate A) | x (in %) | −0.10 | −0.18 | −0.36 | −1.03 | −2.05 |
| | s | 0.01 | 0.00 | 0.00 | 0.01 | |
| laminate B) | x (in %) | −0.09 | −0.16 | −0.31 | −0.91 | −1.81 |
| | s | 0.00 | 0.00 | 0.01 | 0.01 | |
| laminate B)* | x (in %) | −0.08 | −0.16 | −0.32 | | −1.89 |
| | s | 0.01 | 0.01 | 0.01 | | |
| Bottle** | x (in %) | | | −0.71 | | −4.25 |
| | s | | | 0.02 | | |

X = mean value of 10 samples
S = standard deviation
*produced on a new production line
**Reference bottle: HDPE-bottle with diameter 22 mm, height 45 mm and a wall-thickness of 0.8 mm

TABLE 5
(RT at 35% rh):

| | | 7 | 14 | 1 | 3 | extrapolation |
|---|---|---|---|---|---|---|
| Sample | | days | days | month: | months | 24 months: |
| laminate A) | x (in %) | −0.02 | −0.04 | −0.07 | −0.22 | −1.73 |
| | s | 0.01 | 0.01 | 0.01 | 0.03 | |
| laminate B) | x (in %) | −0.02 | −0.03 | −0.06 | −0.18 | −1.43 |
| | s | 0.01 | 0.01 | 0.01 | 0.01 | |
| laminate B)* | x (in %) | −0.02 | −0.03 | −0.06 | | −1.45 |
| | s | 0.00 | 0.00 | 0.00 | | |
| Bottle** | x (in %) | | | −0.15 | | −3.59 |
| | s | | | 0.05 | | |

X = mean value of 10 samples
S = standard deviation
*produced on a new production line
**Reference bottle: HDPE-bottle with diameter 22 mm, height 45 mm and a wall-thickness of 0.8 mm

The WVTR measurements as well as the weight loss measurements show improved properties of the inventive tubes over a standard bottle.

While there are shown and described presently preferred embodiments of the invention, it is to be distinctly understood that the invention is not limited thereto but may be otherwise variously embodied and practiced within the scope of the following claims.

1. Compressible tube comprising a tube body (1), a shoulder (2a, 2b) and an applicator, said applicator being suitable for applying a tube content in dosed droplets, said applicator comprising a sterile venting valve and said tube body having a restoring force (R) sufficient to essentially restore the original volume of the tube body after each of a predetermined number of applications.

2. The compressible tube of claim 1, wherein the restoring force (R) is such that the original volume is restored to at least 85%, preferably at least 90%, more preferred at least 95% for the number of applications possible until the container is empty.

3. The compressible tube of claim 1, wherein the tube body is a laminate (4) composed of three layers or foils or films (5, 7, 8), an outside layer or foil or film (5), a center layer or foil or film (7), and an inner layer or foil or film (8), wherein
the outside layer or foil or film (5) contains at least about 85% polyethylene composed of HDPE and LDPE, or HDPE and LLDPE, wherein the HDPE content is at least about 70%, preferably at least about 80%, more preferably at least about 85%, much preferably about
90%, and the outside layer or foil or film (5) optionally comprises up to 15% additives and/or pigments for esthetic purposes, wherein the additives are preferably selected from light barrier additives and light stabilizers,
the center layer or foil or film (7) is a non-oriented HDPE layer or foil or film containing at least about 85%, preferably at least about 90% HDPE, optionally provided with a barrier coating (10) such as a metallization, and
the inner layer or film or foil (8) is a mono foil or a coextruded foil comprising a coextruded EVOH barrier layer (9),
wherein
the mono layer or film or foil is a HDPE/LDPE or HDPE/LLDPE foil with a HDPE content of at least about 70%, and
the coextruded layer or film or foil comprising an EVOH layer comprises at least about 70% polyethylene of the same composition as the mono foil and an EVOH layer of a thickness of at most 30 μm, more preferred at most 20 μm, most preferred about 9 μm, as well as tie layers (6a) of maleic anhydride grafted LLDPE arranged between the EVOH layer and a polyethylene layer in a thickness each that is similar to or up to about 50% smaller than the thickness of the EVOH layer.

4. The compressible tube of claim 3, wherein the outside layer or foil or film (5) and the center layer or foil or film (7) are connected via a tie layer (6), preferably a extruded tie layer (6), and the center layer or foil or film (7) and the inner layer or foil or film (8) are also connected via a tie layer (6), preferably a extruded tie layer (6).

5. The compressible tube of claim 4, wherein at least one tie layer (6) contains at least about 90%, preferably about 95%, more preferably 100% HDPE.

6. The compressible tube of claim 3, wherein the HDPE content of the tube body is at least 70%.

7. The compressible tube of claim 4, wherein
the outside layer/foil/film (5) has a thickness from 100 μm to 140 μm, in particular about 120 μm;
the tie layer (6) if present has a thickness from 20 μm to 30 μm, in particular about 25 μm;
the center layer/foil/film (7) has a thickness from 60 μm to 100 μm, in particular about 80 μm; and
the inner layer/foil/film (8) has a thickness from 80 μm to 120 μm, in particular about 100 μm.

8. The compressible tube of claim 4, wherein
the laminate (4) has a structure of
outside layer/foil/film (5) in a thickness from 100 μm to 140 μm, in particular about 120 μm
tie layer (6) in a thickness from 20 μm to 30 μm, in particular about 25 μm
center layer/foil/film (7) in a thickness from 60 μm to 100 μm, in particular about 80 μm
tie layer (6) in a thickness from 20 μm to 30 μm, in particular about 25 μm
inner layer/foil/film (8) in a thickness from 80 μm to 120 μm, in particular about 100 μm and a layer composition of
outside layer/foil/film (5) 90/10 HDPE/LDPE or HDPE/LLDPE
center layer/foil/film (7) 100 HDPE
inner layer/foil/film (8) 80/20 HDPE/LDPE or HDPE/LLDPE, or a layer/foil/film with HDPE/LLDPE 80/20 inside/outside plus 9 μm EVOH layer (9) and 2×7 μm tie layer (6a)
tie layer (6) 100 HDPE.

9. The compressible tube of claim 1, wherein the longitudinal seam of the tube body extends decentralized from an end seam (3) to the shoulder (2a, 2b).

10. The compressible tube of claim 1, wherein the container has a volume of 10 ml, a diameter of 22 mm and the restoring force is 63 mBar.

11. The compressible tube of claim 1, wherein the applicator has a vented cap.

12. The compressible tube of claim 1 that contains a pharmaceutical composition for ophthalmic use.

13. A laminate (4) for producing the tube body (1) of claim 1, wherein the laminate (4) has the following structure
outside layer/foil/film (5) in a thickness from 100 μm to 140 μm, in particular about 120 μm
tie layer (6) in a thickness from 20 μm to 30 μm, in particular about 25 μm
center layer/foil/film (7) in a thickness from 60 μm to 100 μm, in particular about 80 μm
tie layer (6) in a thickness from 20 to 30 μm, in particular about 25 μm
inner layer/foil/film (8) in a thickness from 80 μm to 120 μm, in particular about 100 μm
wherein
the outside layer/foil/film (5) contains at least 85% polyethylene composed of HDPE and LDPE, or HDPE and LLDPE, wherein the HDPE content is at least 70%, preferably at least 80%, more preferably at least 85%, much preferably about 90%, and the outside layer or foil or film (5) optionally comprises up to 15% additives and/or pigments for esthetic purposes, wherein the additives are preferably selected from light barrier additives and light stabilizers,
the center layer/foil/film (7) is a non-oriented HDPE layer or foil or film containing at least about 85%, preferably at least about 90% HDPE, optionally provided with a barrier coating (10) such as a metallization,
the inner layer/film/foil (8) is a mono foil or a coextruded foil comprising a coextruded EVOH barrier layer (9),
wherein
the mono layer or film or foil is a HDPE/LDPE or HDPE/LLDPE foil with a HDPE content of at least about 70%, and
the coextruded layer/film/foil comprising an EVOH layer comprises at least about 70% polyethylene of the same composition as the mono foil and an EVOH layer of a thickness of at most 30 μm, more preferred at most 20 μm, most preferred about 9 μm, as well as tie layers (6a) of maleic anhydride grafted LLDPE arranged between the EVOH layer and a polyethylene layer in a thickness each that is similar to or up to about 50% smaller than the thickness of the EVOH layer,
the tie layer (6) contains at least 90%, preferably 95%, more preferably 100% HDPE.

14. The laminate (4) according to claim 13, having the following structure
outside layer/foil/film (5) in a thickness from 100 μm to 140 μm, in particular about 120 μm
tie layer (6) in a thickness of from 20 μm to 30 μm, in particular about 25 μm
center layer/foil/film (7) in a thickness from 60 μm to 100 μm, in particular about 80 μm
tie layer (6) in a thickness of from 20 μm to 30 μm, in particular about 25 μm
inner layer/foil/film (8) in a thickness from 80 μm to 120 μm, in particular about 100 μm and a layer composition
outside layer/foil/film (5) 90/10 HDPE/LDPE or HDPE/LLDPE
center layer/foil/film (7) 100 HDPE
inner layer/foil/film (8) 80/20 HDPE/LDPE or HDPE/LLDPE, or a layer/foil/film with HDPE/LLDPE 80/20 inside/outside plus 9 μm EVOH layer (9) and 2×7 μm tie layer (6a)
tie layer (6) 100 HDPE.

15. Use of a compressible tube of claim 1 for being filled with a pharmaceutical composition for ophthalmic use.
